(19) United States
(12) Patent Application Publication
(10) Pub. No.: US 2010/0318125 A1
(43) Pub. Date: Dec. 16, 2010

(54) Anchor Device For Knot-Free Attachment Of Tissue To A Bone

(76) Inventors: Christian Gerber, Zumikon (CH); Sascha Berberich, Tuttlingen (DE); Rainer Ulmschneider, Tuttlingen (DE)

Correspondence Address: ST. ONGE STEWARD JOHNSTON & REENS, LLC, 986 BEDFORD STREET, STAMFORD, CT, 06905-5619, US

(21) Appl. No.: 12/760,316

(22) Filed: Apr. 14, 2010

(30) Foreign Application Priority Data
Apr. 15, 2009 (DE) 10 2009 018 136.9

(51) Int. Cl. A61B 17/04

(52) U.S. Cl. 606/232

(57) ABSTRACT

An anchor device serves for knot-free attachment of a tissue to a bone by means of at least one suture threaded through that anchor device. The device comprises an anchor body and a transverse bore for receiving a suture passing through said anchor body in a distal end region. The anchor body is provided with a longitudinal bore extending axially from a proximal end up to said transverse bore, and an axially displaceable clamping element is held in said longitudinal bore. An expanding element is provided for radially expanding at least one section of a wall surrounding said longitudinal bore.

### BACKGROUND OF THE INVENTION

The invention relates to an anchor device for a knot-free attachment of tissue to a bone by means of at least one suture threaded through the anchor device.

Such an anchor device has been disclosed in DE 10 2006 010 116 A1. The known device has a transverse bore arranged in the distal end region of an anchor body and extending through the latter for threading the at least one suture transversely through the anchor body. A longitudinal bore in the anchor body is surrounded by a wall and extends up to the transverse bore. An axially displaceable clamping element, which is held in the longitudinal bore, serves for clamping the suture threaded through the anchor body between the clamping element and the anchor body.

Such anchor devices, also referred to as suture anchors, are used in the field of medicine for reattaching tissue, usually tendons, that detached from a bone to the bone.

For this, the anchor device, together with a suture threaded through the anchor device, is in principle driven into the bone such that it is secured. On one side, the suture is connected to the detached tissue and, on the other side, it is clamped in the anchor device, and this results in the detached tissue being attached to the bone. This can be done without knotting the suture to avoid upstanding knots. This is known as knot-free attachment of a tissue to a bone.

The anchor body of approximately cylindrical shape of DE 10 2006 101 116 A1 has projections being provided on the outside of said device in order to prevent withdrawal of the anchor device after it has been inserted into the bone. These projections are designed as barb-like elements. When a tensile load occurs, they develop a resistive force countering withdrawal of the anchor element from the bone after the anchor body was punched or driven into the bone and the suture was clamped.

The tissue to be attached is firstly pierced by the suture, usually with the aid of a needle. Then the two free suture ends are threaded through the trans-verse bore of the anchor body. The connection between anchor body and tissue to be attached is brought about in the form of a suture loop. The anchor body, together with the suture, now can be already inserted into the bone and the free suture ends are subsequently pulled, resulting in the protruding loop of the suture connected to the tissue being pulled toward the position to be attached.

The clamping element now is moved in the longitudinal bore in order to fix the relative position between the suture (and the tissue connected thereto) and the anchor body, as a result of which the section of the suture located in the transverse bore is fixed, i.e. clamped, in a certain position on the anchor body. This likewise fixes the loop holding the tissue. The protruding free ends of the suture can then be cut off, for example.

If a strongly charged tendon, e.g. in the region of the shoulder or the region of the knee, is attached, it is obvious that considerable tensile forces act, from the tendon, onto the assembly inserted into the bone. This tensile force may loose the assembly of anchor body, clamping element and suture clamped therebetween.

The barbs protruding on the outer side of the anchor body occupy a certain space in the bone. Compared to an anchor body without projections, the opening in the hone is expanded by the projections when driving the anchor body into the bone. After the anchor element is driven in, the projections only generate little pressure on the sidewall of the created opening.

The interaction between the barbs and the bone material is not intensive. This results in an insufficient and unsatisfactory anchoring of the anchor device in the bone.

It is therefore an object of the present invention to create an anchor device of the type mentioned at the outset, which has a simple design and, in particular, ensures effective and reliable anchoring of the anchor device.

### SUMMARY OF THE INVENTION

According to the invention, this object is achieved by an anchor device comprising an anchor body having a proximal and a distal end, a transverse bore passing said anchor body in a distal end region of said anchor body, said transverse bore serving for threading at least one suture transversely through said anchor body, said anchor body being provided with a longitudinal bore extending axially from said proximal end to said transverse bore, said longitudinal bore being surrounded by a wall of said anchor body, and an axially displaceable clamping element hold in said longitudinal bore, said clamping element clamps said at least one suture in said transverse bore when displaced in a direction of said distal end of said anchor body, and wherein an expanding element for radially expanding at least one section of said wall surrounding said longitudinal bore is provided.

The body of the anchor can be driven into a core-hole bore in the bone, the clear internal diameter of said bore approximately corresponding to the external diameter of the body. After pulling the tissue via the thread and followed by clamping the suture with the clamping element, the tissue is attached to the bone surface. The expanding element expands the wall radially. As a result, the diameter of the wall in the expanded region thereof becomes larger than the diameter of the core-hole bore. The wall section that can be expanded is driven radially into the bone material and there it locks or blocks withdrawal of the anchor from the bone.

The body of the anchor can have a smooth external side or the latter can be provided with circumferential ribs. What is decisive is that one section of the wall has moved into the bone material after expanding so that it blocks against the withdrawal thereof.

The make-up of bones is such that the outer surface (cortical bone) is relatively hard and the region lying deeper inside (spongy bone) is relatively soft. If the anchor is driven in so far that the expanded wall region lies under the hard bone cortex, said hard bone region additionally blocks withdrawal of the anchor. The wall region that can be expanded penetrates far into the spongy bone.

In a refinement of the invention, the expanding element is formed on the clamping element.

This measure reduces the number of components, lessens the production complexity and simultaneously simplifies handling and assembly. This affords the possibility of performing both the expanding of the wall and the clamping of the suture using one and the same element. This can be performed simultaneously, i.e. when clamping the at least one suture in the transverse bore the expansion of the wall section takes place via the expanding element.

In a further refinement of the invention, the expanding element is arranged on the proximal end region of the clamping element.

The advantage of this measure is that the clamping element can be inserted from the proximal end of the anchor body in the direction of the distal end, and can firstly be prepared for its actual object, namely to clamp the suture. The expanding element arranged at the proximal end is only used once the clamping element has been almost completely introduced into the longitudinal bore.

In a further refinement of the invention, the expanding element has a radially extended section, the external diameter of which being greater than the clear internal diameter of the longitudinal bore.

This measure offers a simple and efficient option for providing a radially outward expanding force acting on the section to be expanded as a result of the axial movement of the expanding element, even in the case of small components.

In a further refinement of the invention, the radially extended section merges into a section with a smaller diameter via a bevel.

The expanding element can have the shape of a conical wedge, as a result of which the wedge action is used to expand the at least one section of the wall that can be expanded radially outward. As a result, the expanding movement is harmonious and not jerky.

In a further refinement of the invention, one wall section, engaging with the radially extended section when expanded, has a corresponding complementary bevel.

This measure constitutes an operationally secure and effective option for transmitting force from the expanding element onto the wall section that can be expanded.

In a further refinement of the invention, the longitudinal extension of the clamping element and the position of the expanding element on the clamping element are selected such that the expanding element then can be brought into expanding engagement with the wall section that can be expanded when a distal end region of the clamping element is brought into clamping engagement with the suture.

The advantage of this measure is that the clamping process and the expanding process are optimally matched to one another, and so the clamping and expanding processes are undertaken simultaneously and in one moving step when the clamping element, on which the expanding element is formed, is moved to the distal end. As a result of which the handling is significantly simplified.

In a further refinement of the invention, the wall section that can be expanded is defined by at least one incision into the wall.

The provision of incisions can precisely define the sections that can be expanded and their ability to expand can be significantly increased. This realizes defined expanding with, at the same time, less expanding force needing to be applied.

In a further refinement of the invention, there are a plurality of incisions in the wall, which incisions are distributed over the circumference and run in the axial direction.

This measure provides, distributed over the circumference, a plurality of expandable wall sections, resulting in further improved anchoring of the anchor device via said expanded wall section biting into the bone material.

In a further refinement of the invention, said incisions extend from the proximal end of the wall in the direction toward the transverse bore.

On the one hand, from a production point of view, the advantage of such a refinement of the anchor body is that the incisions can be produced with very little effort. On the other hand, the wall sections that can be expanded have a free edge on the proximal end, which free edge can bite its way into the bone material after expanding and can provide enormous resistance in the case of a tensile force.

In a further refinement of the invention, said at least one wall section that can be expanded has a hinge extending around a circumference of the anchor body.

The pivoting movement of a wall section runs along the hinge in a defined fashion.

This measure additionally benefits the ability to expand of the sections that can be expanded to the extent that the material load on the section that can be expanded is minimized at the connection point to the remainder of the wall and, moreover, the expanding force to be applied is reduced further. Thus, the possibility of the wall sections that can be expanded breaking due to material loading or material fatigue is reduced.

In a further refinement of the invention, said hinge extends between two incisions running in the axial direction.

The significant advantage offered by this measure is that a wall section determined by two neighbored incisions undergoes a defined expanding movement due to the hinge profile.

In a further refinement of the invention, the hinge extends over the entire circumference.

The advantage of this measure is that the entire circumference of the wall can be used by sections that can be expanded, resulting in further optimization of the anchoring.

In a further refinement of the invention, the hinge is designed as a film hinge.

From a production point of view, the advantage of this measure is that the hinge can for example be designed as a circumferential recess or can already be produced during injection moulding in the case of plastics, resulting in significantly reduced production costs.

In a further refinement of the invention, the clamping element is designed as a screw, which can be screwed into a female thread in the longitudinal bore, and the expanding element is designed as an enlarged screw head.

Such a refinement of the anchor device offers a simple, easily manageable, cost-effectively producible and efficient implementation of the anchoring of the anchor body and the fixing of the suture. This allows absorption of significant tensile forces in the case of anchors with dimensions of the order of a few millimeters. Consideration of course has to be given to the fact that there already is a lesion on the bone due to the tear of the tendon to be attached. Should it now be possible to bring about the anchoring with an anchor element that is as small as possible in an effective fashion, this is very advantageous for the healing process. The combined clamping/expanding device permits such small designs.

It is understood that the aforementioned features and the features yet to be explained below can be used not only in the specified combinations, but also in other combinations or on their own, without departing from the scope of the present invention.

### BRIEF DESCRIPTION OF THE DRAWINGS

The invention will be described in more detail and explained in the following text on the basis of a few selected exemplary embodiments in conjunction with the attached drawings, in which:

FIG. 1 shows an exploded view of an anchor device according to the invention and the clamping element thereof, in which the handling tool for placing the anchor device into a bone is illustrated at the top end,

FIG. 2 shows a perspective view of the clamping element in FIG. 1,

FIG. 3 shows a sectional view of the anchor body in FIG. 1 along the line III-III in FIG. 1,

FIG. 4 shows a perspective view of the anchor body in FIG. 1 and FIG. 3,

FIG. 5 shows a situation when placing the anchor element before assembling the components,

FIG. 6 shows a situation in which the clamping element is being screwed into the anchor element,

FIG. 7 shows a situation in which the clamping element just encounters the suture and the expanding element engages with the wall to be expanded,

FIG. 8 shows the situation with a clamped suture and expanded wall section,

FIG. 9 shows a detailed view of the contact point between the anchor device and the bone from FIG. 7, in which the expanding element arranged on the clamping element is just engaging with the section that can be expanded,

FIG. 10 shows the expanding process following FIG. 9, which shows how the wall section that can be expanded bites into the bone, and

FIG. 11 shows a sectional view after placing the anchor device, attaching the tissue by means of the suture and fixing the anchor device by means of the expanding element in the bone.

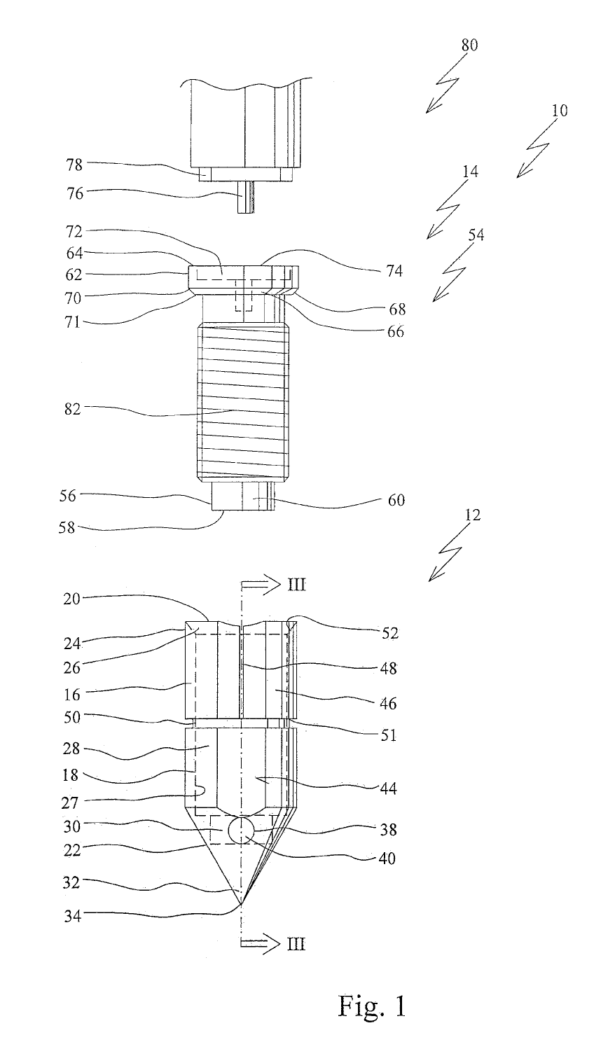

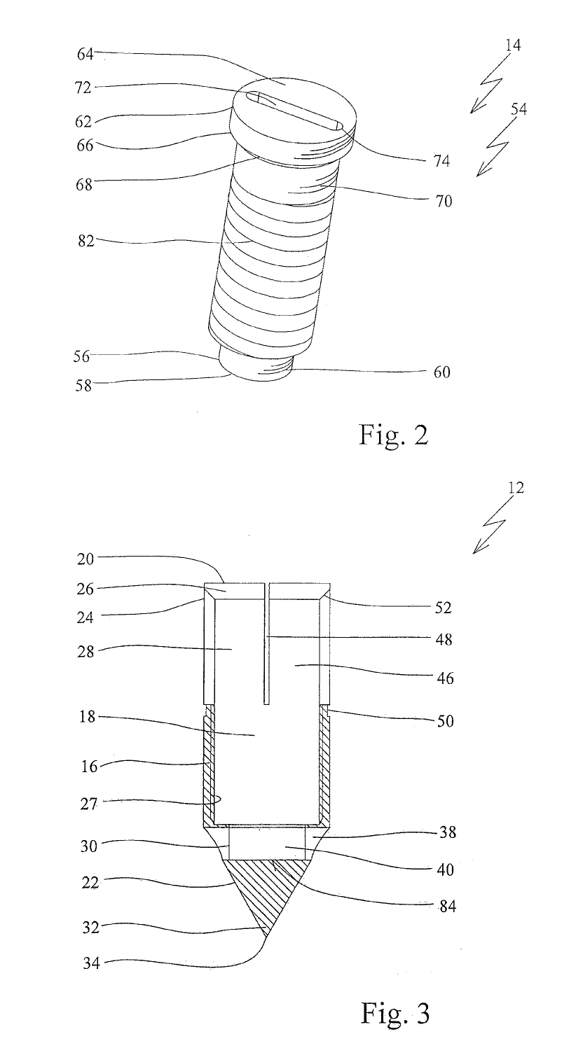

### DETAILED DESCRIPTION OF A PREFERRED EMBODIMENT

An anchor device, illustrated in the figures, for knot-free attachment of tissue to a bone by means of at least one suture threaded through the anchor device is denoted in its entirety by the reference sign 10.

The anchor device 10 has an anchor body 12 and a clamping element 14.

The anchor body 12 has an elongate, approximately cylindrical shape with a wall 16 surrounding and a longitudinal bore 18. The longitudinal bore 18 is designed as a blind-hole bore and it extends from the proximal end 20 of the anchor body 12 to the distal end region 22 of the anchor body 22. The longitudinal bore 18 has a first longitudinal bore section 26 arranged at the proximal end region 24 of the anchor body 12, a second longitudinal bore section 28 adjacent thereto, extending to the distal end region 22 and having a female thread 27, and a third longitudinal bore section 30 adjacent thereto, arranged at the distal end region 22 and having a smaller diameter than the second bore section 28.

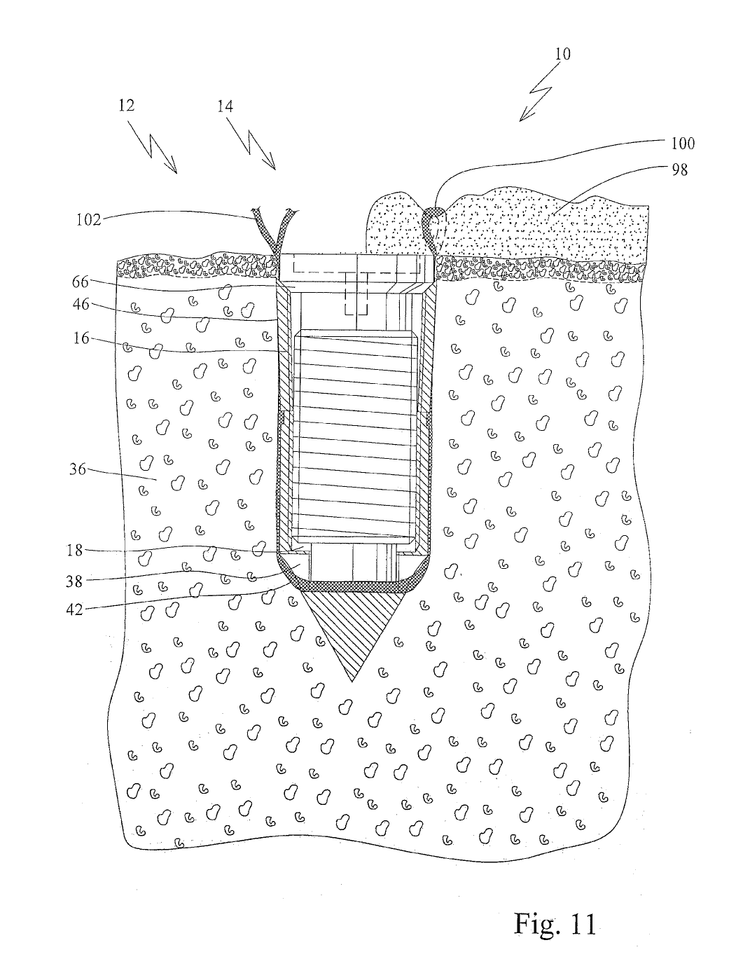

Additionally, the anchor body 12 has a conical design at its distal end region 22 and has an anchor body tip 34 at its distal end 32. The anchor body tip 34 is used to place the anchor body 12 onto a bone 36 (see FIG. 11), and insert it therein in a precise fashion and with only little force expenditure due to the splitting effect.

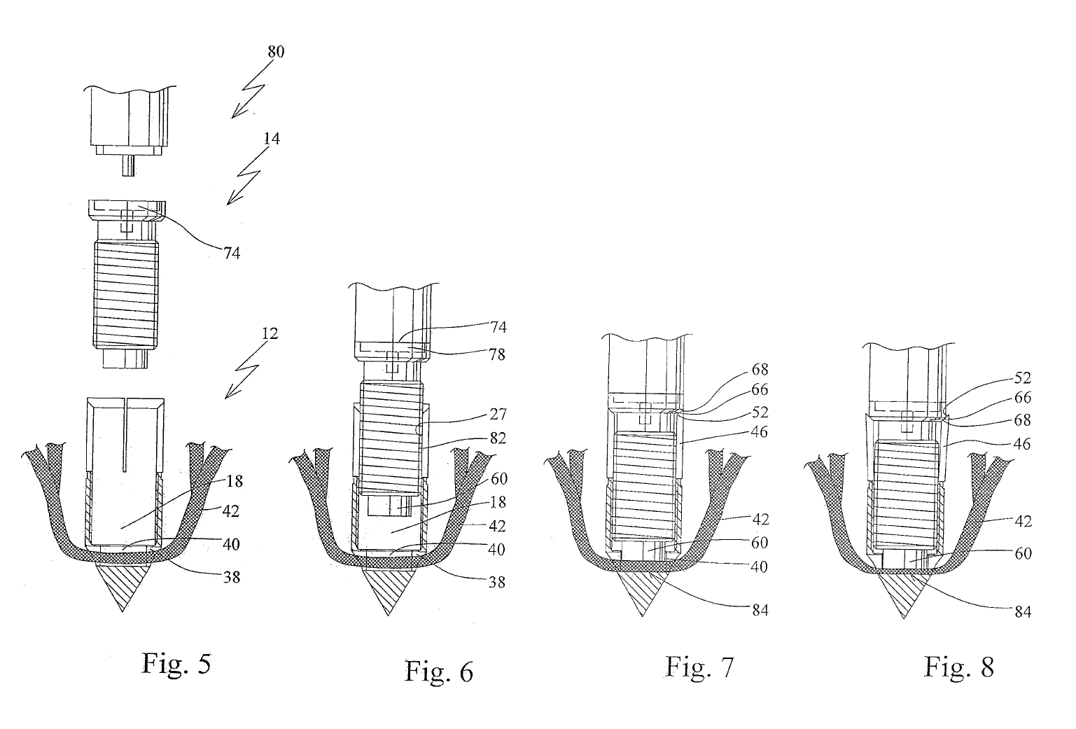

Furthermore, the anchor body 12 has a transverse bore 38 passing transversely through it in its distal end region 22. The transverse bore 38 passes through the third longitudinal bore section 30 of the longitudinal bore 18. Here, the common section of section of the transverse bore 38 and the third longitudinal bore section 30 is defined as a clamping chamber 40. The transverse bore 38 serves to receive a section of a suture 42 (see FIG. 5), the latter being fixed in the clamping chamber 40 by the clamping element 14, as explained in detail in the following text relating to the description of FIG. 5 to FIG. 8.

Two grooves 44, arranged opposite to one another at the outer face of anchor body 12 are respectively arranged directly above the openings of the transverse bore 38 and are used to guide the suture 42 in the direction of the proximal end 20 of the anchor body 12 on respectively both sides after said suture has passed through the transverse bore 38.

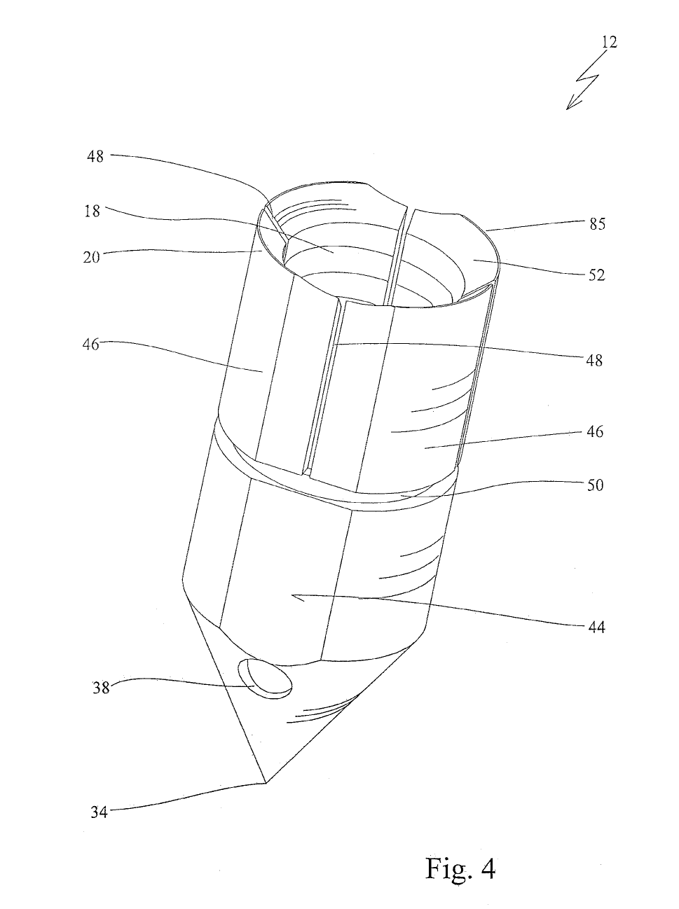

FIG. 4 shows that the anchor body 12 has arranged four expandable sections 46 in the proximal end region 24. The sections 46 that can be expanded are evenly distributed around the circumference of the wall 16 of the anchor body 12 and are defined by four axial incisions 48, which extend axially from the proximal end 20 in the direction toward the transverse bore 38.

A hinge 50 at a lower end of each wall section 46 serves as an elastic, rotatable linkage of the sections 46 with the remaining wall 16 only a small expanding force has to be applied for expanding the sections 46. The hinge 50 is designed as a recess 51, which is arranged at the lower end of the incisions 48 and extends over the entire circumference of the wall 16.

The sections 46 that can be expanded inwardly have a tapered rim at the proximal end 20 and have an internal bevel 52 sloping in the distal direction in the region of the first longitudinal bore section 26 of the longitudinal bore 18. The function of this internal bevel 52 will be explained in more detail in the following figures.

The clamping element 14 designed as a screw 54 has a clamping section 60 which is arranged at the distal end region 56 of said clam element 14. The clamping section 60 is designed as a cylindrical end plug 58 and is designed to penetrate the clamping chamber 40 in order to fix the suture 42. Furthermore, the clamping element 14 has a expanding element 66, which is arranged in the proximal end region 62 of said clamping element 14 and designed as an enlarged screw head 64. Here, the expanding element 66 has an external bevel 68, which runs over the entire circumference and corresponds to the internal bevel 52 of the sections 46 that can be expanded, transitioning from a radially extended section 70 to a section 71 with a smaller diameter. As will be shown in more detail below, the external bevel 68 interacts with the internal bevel 52 in a wedge-like fashion in the case of an axial movement of the expanding element 66 and thereby simultaneously expands the expanding sections 46 radially outward.

The screw head 64 has a groove 72 serving as a tool holder 74. The groove 72 is designed such that it can hold a cylindrical centring pin 76 and a projection 78 of a driving-in unit 80, so that a moment can be transferred in an interlocking fashion. Here, the centring pin 76 is merely used as centring aid when the driving-in unit 80 engages in the screw head 64 and does not contribute to the transfer of moments.

The clamping element 14 additionally has a male thread section 82 extending between the screw head 64 and the cylindrical end shaft 58. The thread section 82 is the place of connection between the clamping element 14 and anchor body 12 and thus transmits the clamping force from the clamping element 14 onto the suture 42 and the expanding force from the expanding element 66 to the sections 46 that can be expanded when it is screwed into the female thread 27 of the anchor body 12.

FIG. 3 shows that the clamping chamber 40 has a clamping base 84 against which the suture 42 is pushed and thus fixed by means of the clamping section 60 of the clamping element 14.

It should be noted that the clamping base 84 can have any shape corresponding to the clamping section 60 of the clamping element 14, with however the clamping base 84 having a flat design and the clamping section 60 likewise having a flat design with a chamfered edge being preferable, and so the clamping force is transmitted in a planar fashion onto the suture 42 from the clamping element 14 via the clamping section 60. This substantially reduces the risk of damage to the suture 42 due to punctiform transmission of force, as is the case in, for example, strongly curved shapes.

FIG. 4 shows that the four sections 46 that can be expanded are formed by four incisions 48 reaching through the wall 16 and running in the axial direction. Here, the incisions 48 extend from the proximal end 20 of the anchor body 12 to the recess 50 arranged approximately level with the middle of the anchor body 12.

In order to explain the function of the anchor device 10, FIG. 5 to FIG. 8 show the procedure of the operation technique for preparing the anchor device 10 until the latter is attached in the bone.

FIG. 5 shows an exploded view including the anchor body 12, the clamping element 14 arranged above the longitudinal bore 18 and the driving-in unit 80 arranged above the tool holder 74. Here, the suture 42 is threaded twice through the transverse bore 38 and thus passes through the clamping chamber 40. FIG. 11 shows that the suture 42 crosses the transverse bore 38 twice, i.e. respectively once in opposing directions, and so a loop is created on one side previously connected to the tissue to be attached, for example by means of a needle hole. Since the two suture sections lie behind one another in the anchor body, only one suture section can be seen in the figure sequence of FIGS. 5-8.

FIG. 6 shows a subsequent snapshot, in which the projection 78 of the driving-in unit 80 engages with the tool holder 74 of the clamping element 14 and the clamping element 14 is screwed into the longitudinal bore 18 with the female thread 27 by means of the thread section 82. In the process, the clamping section 60 approaches the clamping chamber 40 and thus the suture 42 to be clamped.

The snapshot shown in FIG. 7 represents the time at which the clamping section 60 engages in a clamping fashion with the suture 42 and the expanding element 66 engages in a expanding fashion with the sections 46 that can be expanded, i.e. the beginning of the clamping and expanding process.

What is shown is that at this time, when the clamping section 60 reaches the suture 42 and presses it against the clamping base 84, the internal bevel 52 of the expanding element 66 simultaneously engages with the external bevel 68 of the wall sections 46 that can be expanded.

FIG. 8 shows the final step, i.e. fixing the suture 42 and anchoring the anchor device. While the clamping section 60 presses the suture 42 against the clamping base 84 and thereby fixes it as a result of the axial movement of the clamping element 14, the expanding element 66 interacts with the sections 46 that can be expanded in a expanding fashion. As explained above, the external bevel 68 of the expanding element 66 engaging in the corresponding internal bevel 52 of the wall sections 46 that can be expanded generates a wedge effect, which simultaneously expands the wall sections 46 that radially outward, resulting in the anchor device 10 being anchored in the bone 36.

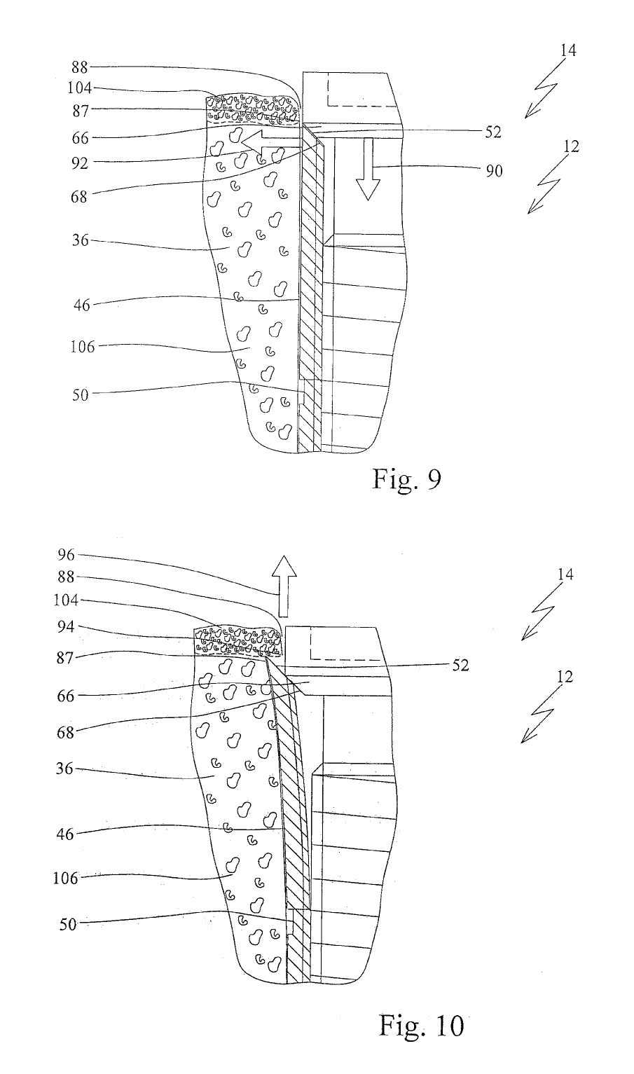

FIG. 9 and FIG. 10 are detailed views similar to FIG. 7 and FIG. 8, respectively, and show the expanding element 66 engaging with a wall section 46 that can be expanded in a expanding fashion and the wall section 46 biting into a sidewall 87 of an opening 88 in the bone 36.

FIG. 9 shows that the anchor body 12 was driven so far into a previously effected core-hole bore that the proximal end comes to rest below the relatively hard bone cortex 104. The bone material 106 is significantly softer (spongy) deeper inside the cortex 104.

FIG. 9 illustrates a snapshot at the beginning of the expanding process. Here, the axial movement direction of the clamping element 14 is illustrated by means of the arrow 90. In the process, the expanding or radial force generated due to the wedge effect generates an expanding movement of the wall section 46 as the expanding element 66 penetrates further into the longitudinal bore 18, the movement direction of which section is illustrated by an arrow 92.

FIG. 10 shows how the anchor device 10 expands in this bone region due to the expanded wall section 46, and how the wall section 46 penetrates into the sidewall 87 of the opening 88, particularly in the region of the proximal end 20, and bites into the relatively soft spongeous material 106 of the bone 36 and thereby fixes the anchor device 10 in the opening 88 of the bone 36. The proximal region of the expanded wall section 46 now comes to rest below the hard bone cortex 104. As a result of this, the bone cortex 104 additionally blocks a withdrawal of the anchor body 12. The widening of the anchor body 12 in the region of the wall sections 46 accordingly leads to an increase in the pressure on the sidewall 87 of the opening 88, which in turn leads to a significant increase in the resistance.

FIG. 10 furthermore shows that the internal bevel 52 of the wall section 46 has an additional function. While the internal bevel 52 firstly interacts with the external bevel 68 of the sharp upper rim of the expanding element 66, it secondly increases the biting into the bone 36 due to its sharp upper rim shape, as a result of which the anchoring of the anchor device 10 is significantly increased. In the case of a tensile load, illustrated by an arrow 96, additional blocking and hence an increased resistance is provided by the expanded wall sections 46.

Finally, FIG. 11 shows a completely placed anchor device 10, fixing a tissue 98, e.g. a tendon, by means of the suture 42, fixing the suture 42 by means of the clamping element 14 and anchoring the anchor device 10 in the opening 88 in the bone 36 by means of the extended wall section 46.

What is shown here is that the tissue 98 is held by a loop 100 formed by the suture 42, which loop partly penetrates the tissue 98. Two suture ends 102 should clarify that the excess suture section of the suture 42 is cut after fixing the suture 42. No upstanding knot disturbs the healing process.

1. An anchor device for a knot-free attachment of a tissue to a bone by means of at least one suture threaded through the anchor device, comprising
an anchor body having a proximal and a distal end
a transverse bore passing said anchor body in a distal end region of said anchor body, said transverse bore serving for threading at least one suture transversely through said anchor body,
a longitudinal bore in said anchor body extending axially from said proximal end to said transverse bore, said longitudinal bore being surrounded by a wall of said anchor body,
an axially displaceable clamping element held in said longitudinal bore, said clamping element clamps said at least one suture in said transverse bore when displaced in a direction of said distal end of said anchor body, and
an expanding element for radially expanding at least one section of said wall surrounding said longitudinal bore.

2. The anchor device of claim 1, wherein said expanding element is formed on said clamping element.

3. The anchor device of claim 2, wherein said expanding element is arranged on a proximal end region of said clamping element.

4. The anchor device of claim 3, wherein said expanding element has a radially extended section, an external diameter of which section being greater than a clear inner diameter of said longitudinal bore.

5. The anchor device of claim 4, wherein said radially expanded section merges into a section of smaller diameter via a bevel.

6. The anchor device of claim 5, wherein said at least one wall section engaging with said radially extending section when expanded has a corresponding complementary bevel.

7. The device of claim 2, wherein a longitudinal extension of said clamping element and a position of that expanding element on said clamping element are selected such that said expanding element is brought into expanding engagement with said at least one wall section when a distal end region of said clamping element is brought into clamping engagement with said at least one suture.

8. The anchor device of claim 1, wherein said at least one wall section that can be expanded radially is defined by at least one incision into said wall.

9. The anchor device of claim 8, wherein a plurality of incisions are provided in said wall, which incisions are distributed over a circumference of said wall and run in a axial direction.

10. The anchor device of claim 8, wherein said incisions extend from a proximal end of said wall in a direction towards said transverse bore.

11. The anchor device of claim 1, wherein said at least one wall section that can be radially expanded has a hinge extending around a circumference of said anchor body.

12. The anchor device of claim 11, wherein said hinge extends between two neighbored incisions defining an expandable wall section therebetween.

13. The anchor element of claim 12, wherein said hinge extends over an entire circumference of said wall.

14. The anchor device of claim 11, wherein said hinge is designed as a film hinge.

15. The anchor element of claim 2, wherein said clamping element is designed as a screw which can be screwed into a female thread in the longitudinal bore, and wherein said expanding element is designed as an enlarged head of said screw.
